(19) United States
(12) Patent Application Publication
(10) Pub. No.: US 2004/0167597 A1
(43) Pub. Date: Aug. 26, 2004

(54) Aneurysm treatment devices and methods

(76) Inventors: Peter D. Costantino, Armonk, NY (US); Craig Friedman, Westport, CT (US); Arindam Datta, Hillsborough, NJ (US); Maybelle Jordan, Potomac, MD (US); Yosef Krespi, New York, NY (US); Daniel Klempner, Bloomfield, MI (US); Ian N. Askill, Colorado Springs, CO (US)

Correspondence Address: ANTHONY H. HANDAL, KIRKPATRICK & LOCKHART, LLP, 599 LEXINGTON AVENUE, 31ST FLOOR, NEW YORK, NY, 10022-6030, US

(21) Appl. No.: 10/692,054

(22) Filed: Oct. 23, 2003

Related U.S. Application Data
(60) Provisional application No. 60/420,555, filed on Oct. 23, 2002.
(60) Provisional application No. 60/471,520, filed on May 15, 2003.
(60) Provisional application No. 60/437,955, filed on Jan. 3, 2003.

(51) Int. Cl. A61F 2/06

(52) U.S. Cl. 623/1.1; 623/1.11; 623/1.44

(57) ABSTRACT

An aneurysm treatment device for in situ treatment of aneurysms comprising a collapsible member having a first shape wherein the first shape is an expanded geometric configuration, and a second shape, wherein the second shape is a collapsed configuration that is loadable into a catheter. The aneurysm treatment device is capable of returning to the first shape in the lumen of an aneurysm. Some aneurysm treatment devices comprise a spreadable portion and a projecting portion integral with the spreadable portion. The spreadable portion is capable of resting against and supporting an inner wall of an aneurysm, the projecting portion is capable of being gripped by a surgeon to facilitate insertion and positioning of the device. Other devices have relatively simple shapes and can be implanted to a site as a plurality. Treatment methods are also disclosed.

### STATEMENT REGARDING FEDERALLY SPONSORED RESEARCH OR DEVELOPMENT

(Not applicable.)

### TECHNICAL FIELD

The present invention relates to methods and devices for the treatment of vascular aneurysms and other comparable vascular abnormalities.

### BACKGROUND OF THE INVENTION

The following description of background art may include insights, discoveries, understandings or disclosures, or associations together of disclosures, that were not known to the relevant art prior to the present invention but which were provided by the invention. Some such contributions of the invention may be specifically pointed out below, whereas other such contributions of the invention will be apparent from their context.

The cardio-vascular system, when functioning properly, supplies nutrients to all parts of the body and carries waste products away from these parts for elimination. It is essentially a closed-system comprising the heart, a pump that supplies pressure to move blood through the blood vessels, blood vessels that lead away from the heart, called arteries, and blood vessels that return blood toward the heart called veins. On the discharge side of the heart is a large blood vessel called the aorta from which branch many arteries leading to all parts of the body, including the organs. As the arteries get close to the areas they serve, they diminish to small arteries, still smaller arteries called arterioles and ultimately connect to capillaries. Capillaries are minute vessels where outward diffusion of nutrients, including oxygen, and inward diffusion of wastes, including carbon dioxide, takes place. Capillaries connect to tiny veins called venules. Venules connect to larger veins which return the blood to the heart by way of a pair of large blood vessels called the inferior and superior venae cava. Referring to FIG. 1, arteries 1 and veins comprise three layers known as tunics. An inner layer 2, called the tunica interna, is thin and smooth, constituted of endothelium and rests on a connective tissue membrane rich in elastic and collagenous fibers that secrete biochemicals to perform functions such as prevention of blood clotting by inhibiting platelet aggregation and regulation of vasoconstriction and vasodilation. A middle layer called the tunica media is made of smooth muscle 4 and elastic connective tissue 5 and provides most of the girth of the blood vessel. A thin outer layer 6, called the tunica adventitia, formed of connective tissue secures the blood vessel to the surrounding tissue.

The tunica media 3 differentiates an artery from a vein being thicker in an artery to withstand the higher blood pressure exerted by the heart on the walls of the arteries. Tough elastic connective tissue provides the artery 1 sufficient elasticity to withstand the blood pressure and sudden increases in blood volume that occur with ventricular contractions.

When the wall of an artery, especially the tunica media 3 of that wall, has a weakness, the blood pressure can dilate or expand the region of the artery 1 with the weakness, and a pulsating sac 7 called a berry or saccular aneurysm (FIG. 2), can develop. If the walls of the arteries 1 expand around the circumference of the artery 1, this is called a fusiform aneurysm 8 (FIG. 3) If the weakness causes a longitudinal tear in the tunica media of the artery, it is called a dissecting aneurysm. Saccular aneurysms are common at artery bifurcations 9 (FIGS. 4 and 5) located around the brain. Dissecting aneurysms are common in the thoracic and abdominal aortas. The pressure of an aneurysm against surrounding tissues, especially the pulsations, can cause pain may also cause tissue damage. However, aneurysms are often asymptomatic. The blood in the vicinity of the aneurysm can become turbulent, leading to formation of blood clots, that may be carried to various body organs where they may cause damage in varying degrees, including cerebrovascular incidents, myocardial infarctions and pulmonary embolisms. Should an aneurysm tear and begin to leak blood, the condition can become life threatening, sometimes being quickly fatal, in a matter of minutes.

Because there is relatively little blood pressure in a vein, venous “aneurysms” are non-existent, therefore the description of the present invention is relates to arteries, but applications within a vein, if useful, are to be understood to be within the scope of this invention.

The causes of aneurysms are still under investigation. However, researchers have identified a gene associated with a weakness in the connective tissue of blood vessels that can lead to an aneurysm. Additional risk factors associated with aneurysms such as hyperlipidemia, atherosclerosis, fatty diet, elevated blood pressure, smoking, trauma, certain infections, certain genetic disorders, such as Marfan's Syndrome, obesity, and lack of exercise have also been identified. Cerebral aneurysms occur not infrequently in otherwise healthy and relatively youthful people, perhaps in their early thirties, and have been associated with many untimely deaths.

Aneurysms, widenings of arteries caused by blood pressure acting on a weakened arterial wall, have occurred ever since humans walked the plant. In modern times, many methods have been proposed to treat aneurysms, for example, Greene, Jr., et al., in U.S. Pat. No. 6,165,193 propose a customized compressible foam implant substantially conforming in size and shape with an aneurysm which implant is produced by imaging and modeling the particular aneurysm or other vascular site to be treated. This process is complex and expensive. Other patents disclose introduction of a device, such as a stent or balloon (Naglreiter, et al., U.S. Pat. No. 6,379,329) into the aneurysm, followed by introduction of a hydrogel in the area of the stent to attempt to repair the defect (Sawhney, et al., U.S. Pat. No. 6,379,373).

Still other patents suggest the introduction into the aneurysm of a device, such as a stent, having a coating of a drug or other bioactive material (Gregory, U.S. Pat. No. 6,372,228). Other methods include attempting to repair an aneurysm by introducing via a catheter a self-hardening or self-curing material into the aneurysm. Once the material cures or polymerizes in situ into a foam plug, the vessel can be recanalized by placing a lumen through the plug (Hastings, U.S. Pat. No. 5,725,568).

Another group of patents relates more specifically to saccular aneurysms and teaches the introduction of a device, such as string, wire or coiled material (Boock U.S. Pat. No. 6,312,421), or a braided bag of fibers (Greenhalgh, U.S. Pat. No. 6,346,117) into the lumen of the aneurysm to fill the void within the aneurysm. The introduced device can carry hydrogel, drugs or other bioactive materials to stabilize or reinforce the aneurysm (Greene Jr., et al., U.S. Pat. No. 6,299,619).

Another treatment known to the art comprises catheter delivery of platinum microcoils into the aneurysm cavity in conjunction with an embolizing composition comprising a biocompatible polymer and a biocompatible solvent. The deposited coils or other non-particulate agents are said to act as a lattice about which a polymer precipitate grows thereby embolizing the blood vessel (Evans et al. U.S. Pat. No. 6,335,384).

It is an understanding of the present invention that such methods and devices suffer a variety of problems. For example, if an aneurysm treatment is to be successful, any implanted device must be present in the body for a long period of time, and must therefore be resistant to rejection, and not degrade into materials that cause adverse side effects. While platinum coils may be largely satisfactory in this respect, they are inherently expensive, and the pulsation of blood around the aneurysm may cause difficulties such as migration of the coils, incomplete sealing of the aneurysm or fragmentation of blood clots. If the implant does not fully occlude the aneurysm and effectively seal against the aneurysm wall, pulsating blood may seep around the implant and the distended blood vessel wall causing the aneurysm to reform around the implant.

The delivery mechanics of many of the known aneurysm treatment methods can be difficult, challenging and time consuming.

In light of these drawbacks of the prior proposals, as recognized by the present invention, there is a need for an inexpensive aneurysm treatment that can support and seal the aneurysm, in a manner that will prevent the aneurysm from leaking or reforming.

### SUMMARY OF THE INVENTION

The present invention solves a problem. It solves the problem of providing an aneurysm treatment device and method which is inexpensive and yet can effectively support and seal an aneurysm

To solve this problem, the invention provides an aneurysm treatment device for in situ treatment of aneurysms in mammals, especially humans, which treatment device comprises at least one resiliently collapsible implant collapsible from a first, expanded configuration wherein the implant can support the wall of an aneurysm to a second collapsed configuration wherein the collapsible implant is deliverable into th aneurysm, for example by being loadable into a catheter and passed through the patient's vasculature. Pursuant to the invention, useful aneurysm treatment devices can have sufficient resilience, or other mechanical property, including swellability, to return to an expanded configuration within the lumen of the aneurysm and to support the aneurysm. Preferably, the implant is configured so that hydraulic forces within the aneurysm tend to urge the implant against the aneurysm wall.

It is a feature of the present invention that the implant, or implants if more than one is used, should not completely fill the aneurysm, or other vascular site, as the devices described by Greene Jr. et al are intended to do, but rather, should leave sufficient space within the aneurysm for passage of blood to and preferably around the implant. It is desirable that the implant be designed so that the natural pulsations of the blood can urge blood between the implant and the aneurysm wall to encourage fibroblasts to coat and, if appropriate, to invade the implant.

Because the inventive implants do not have to exactly match the inside topography of the aneurysm, and are producible from low-cost materials, they need not be custom made but can be provided in a range of standard shapes and sizes from which the surgeon or other practitioner selects one or more suitable elements.

It is furthermore preferable that the implant be treated or formed of a material that will encourage such fibroblast immigration. It is also desirable that the implant be configured, with regard to its three-dimensional shape, and its size, resiliency and other physical characteristics, and be suitably chemically or biochemically constituted to foster eventual formation of scar tissue that will anchor the implant to the aneurysm wall.

In a preferred embodiment, the collapsible implant comprises a spreadable portion and a stem-like projecting portion integral with the spreadable portion and can be generally mushroom-shaped or wine glass shaped. The spreadable portion is capable of resting against and supporting an inner wall of an aneurysm, while the projecting portion is capable of being gripped by a surgeon to facilitate insertion and positioning of the device. The spreadable portion may comprise an inner surface and an outer surface, the outer surface being provided with elevations and depression to facilitate blood flow between the inner wall of the aneurysm and the outer surface of the aneurysm treatment device.

A particularly preferred embodiment of the invention comprises a pair of implants which can cooperate to stabilize the aneurysm. To this end, one implant can be seated in the neck of the aneurysm and have a spreading portion spreading into the aneurysm to support the aneurysm wall adjacent the antrum while the other rides in the aneurysm and has a spreading portion supporting the aneurysm wall opposite the neck of the aneurysm. The one implant can be generally wine glass-shaped and the other implant can be generally mushroom-shaped. Such shapes can be modified as appropriate in a given situation.

The aneurysm treatment device is preferably formed essentially entirely, or principally, in so far as concerns its physical structure, from a polymeric foam or a reticulated biodurable elastomeric matrix or the like that is capable of being compressed and inserted into a catheter for implantation. Also, the implant can be formed of a hydrophobic foam having its pore surfaces coated to be hydrophilic, for example by being coated with a hydrophilic material, optionally a hydrophilic foam. Preferably the entire foam has such a hydrophilic coating throughout the pores of the foam.

In one embodiment, the hydrophilic material carries a pharmacologic agent for example elastin to foster fibroblast proliferation. It is also within the scope of the invention for the pharmacologic agent to include sclerotic agents, inflammatory induction agents, growth factors capable of fostering fibroblast proliferation, or genetically engineered an/or genetically acting therapeutics. The pharmacologic agent or agents preferably are dispensed over time by the implant. Incorporation of biologically active agents in the hydrophilic phase of a composite foam suitable for use in the practice of the present invention is described in Thomson U.S. PG PUB 20020018884 more fully identified hereinbelow.

In another aspect, the invention provides a method of treating an aneurysm comprising the steps of:

a) imaging an aneurysm to be treated to determine its size and topography;

b) selecting an aneurysm treatment device according to claim 1 for use in treating the aneurysm; and

c) implanting the aneurysm treatment device into the aneurysm.

Preferably, the method further comprises:

d) loading the aneurysm treatment device into a catheter;

e) threading the catheter through an artery to the aneurysm; and

f) positioning and releasing the aneurysm treatment device in the aneurysm.

Once an aneurysm has been identified using suitable imaging technology, such as a magnetic resonance image (MRI), computerized tomography scan (CT Scan), x-ray imaging with contrast material or ultrasound, and is to be treated, the surgeon chooses which implant he or she feels would best suit the aneurysm, both in shape and size. The one or more implants can be used alone, or the aneurysm treatment device of the invention may also comprise a sheath placed in the lumen of the artery to cover the antrum of the aneurysm. Preferably, the sheath is perforated to permit at least limited blood flow into the aneurysm. The chosen implant or implants are then loaded into an intra-vascular catheter in a compressed state. If desired, the implants can be provided in a sterile package in a pre-compressed configuration, ready for loading into a catheter. Alternatively, the implants can be made available in an expanded state, also, preferably, in a sterile package and the surgeon at the site of implantation can use a suitable device to compress the implant so that it can be loaded into the catheter.

With the implant loaded into the catheter, the catheter is snaked through an artery to the diseased portion of the affected artery using any suitable technique known in the art. Using the catheter the implants are then inserted and positioned within the aneurysm, one at a time if more than one is employed. As the implant is released from the catheter, where it is in its compressed state, it expands and is manipulated into a suitable position whence it can serve the role of supporting the aneurysm. This position may not be the final position which may be attained as a result of movement of the implant by natural forces, notably blood flow.

### BRIEF DESCRIPTION OF THE DRAWINGS

One or more embodiments of the invention and of making and using the invention, as well as the best mode contemplated of carrying out the invention, are described in detail below, by way of example, with reference to the accompanying drawings, in which:

FIG. 1 is a side view of an artery with layers partially cut away to illustrate the anatomy of the artery;

FIG. 2 is a longitudinal cross section of an artery with a saccular aneurysm;

FIG. 3 is a longitudinal cross section of an artery with a fusiform aneurysm;

FIG. 4 is a top view of an artery at a bifurcation;

FIG. 5 is a top view of a artery at a bifurcation with a saccular aneurysm at the point of bifurcation;

FIG. 6 is a side view of an embodiment of an aneurysm treatment implant in accordance with the present invention shaped like a bowl with a flat bottom, having a central projection protruding from the top of the bowl;

FIG. 7 is a top plan view of the embodiment illustrated in FIG. 6;

FIG. 8 is a perspective view of an embodiment in accordance with the present invention shaped like a wine glass, with a base portion, column portion, and bowl portion with substantially convex side walls;

FIG. 9 is a longitudinal cross section of a saccular aneurysm and corresponding artery segment with embodiments of the present invention in an expanded state implanted in a saccular aneurysm;

FIG. 10 is a longitudinal cross section of an artery similar to that illustrated in FIG. 9 further illustrating the addition of a sheath in the lumen of the artery, covering the neck of the aneurysm;

FIG. 11 is a longitudinal cross section of an artery similar to that illustrated in FIG. 9 further illustrating an embodiment of the present invention with ribs;

FIG. 12 is a side view of an embodiment in accordance with the present similar to FIG. 6 wherein the bottom surface of the bowl is rounded;

FIG. 13 illustrates an alternative embodiment of the present invention in the shape of a wine glass having a scaffold-like structure;

FIG. 14 is a perspective view of an embodiment of the present invention similar to FIG. 13 wherein the side walls of the bowl portion are substantially straight;

FIG. 15 is a perspective view of an embodiment of the present invention similar to FIG. 13 wherein a bottom of the bowl portion has an obtuse curvature and little or no side walls;

FIG. 16 is a side view of an embodiment in accordance with the present shaped like a bullet, with sections cut longitudinally;

FIG. 17 is a bottom view of the embodiment of the present invention illustrated in FIG. 16 further illustrating a pattern of the sections;

FIG. 18 is a side view of an alternative embodiment of the present invention similar to the embodiment of FIG. 16 wherein the sections are separated by spaces;

FIG. 19 illustrates an embodiment of the present invention similar to the embodiment of FIG. 18 wherein the top and bottom are mirror images about a plane through the center of the implant;

FIG. 20 is a cross-sectional view of the center portion illustrated in FIG. 19 and viewed along line 20-20 wherein the sections are disposed only around the perimeter;

FIG. 21 is a cross-sectional view of the center portion illustrated in FIG. 19 and viewed along line 20-20 wherein the sections are disposed through the entire cross section of the embodiment; and

FIGS. 22-24 illustrate several embodiments of porous elastomeric implant suitable for employment in the methods or useful as components of the apparatus of the invention.

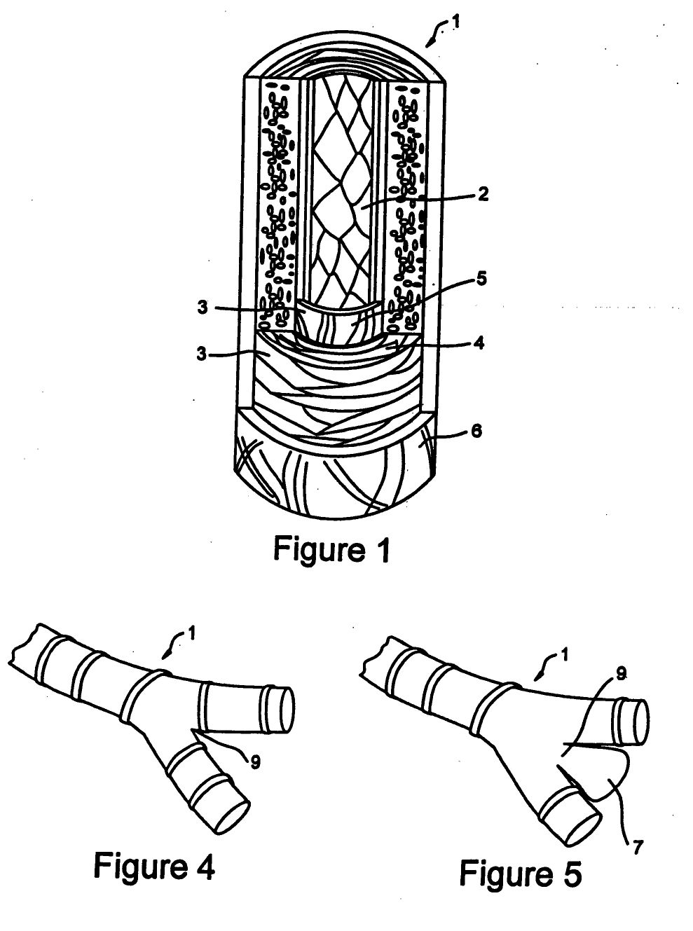

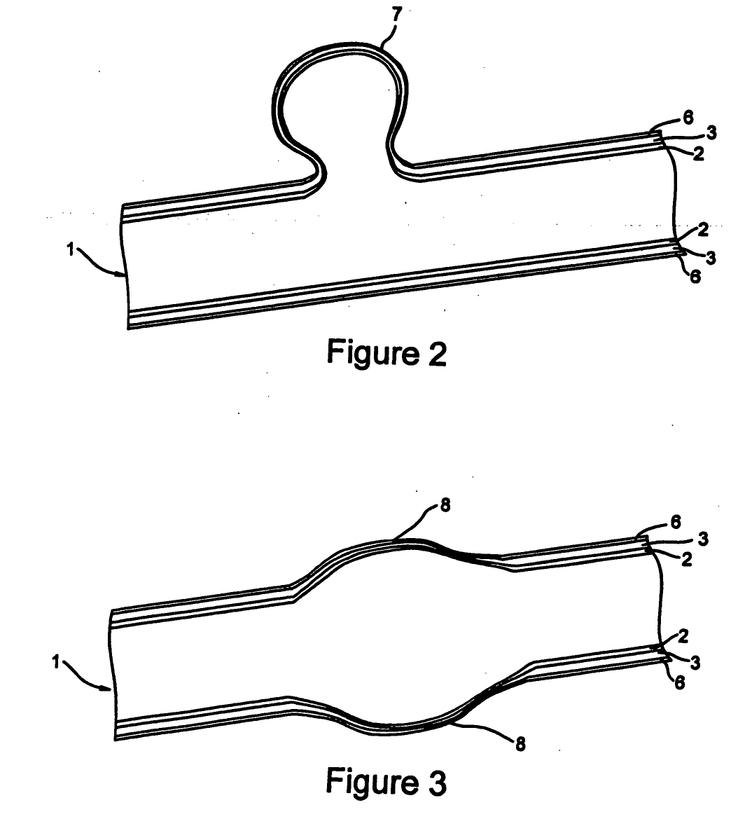

### DETAILED DESCRIPTION OF THE INVENTION

The present invention relates to a system and method for treating aneurysms in situ. As will be described in detail below, the present invention provides an aneurysm treatment device comprising one or more implants designed to be permanently inserted into an aneurysm with the assistance of an intra-vascular catheter. The implants described in detail below can be made in a variety of sizes and shapes. The surgeon being able to choose the best size and shape to treat the patient's aneurysm. Once inserted the inventive aneurysm treatment device is designed to give physical support to the weakened walls of the aneurysm, and reduce or eliminate the pulse pressure exerted on these walls. Furthermore, the inventive aneurysm treatment device can carry one or more of a wide range of beneficial drugs and chemicals that can be released at the affected site for various treatments, such as to aid in healing, foster scarring of the aneurysm, prevent further damage, or reduce risk of treatment failure. By releasing these drugs and chemicals locally, employing the devices an methods of the invention, their systemic side effects are reduced.

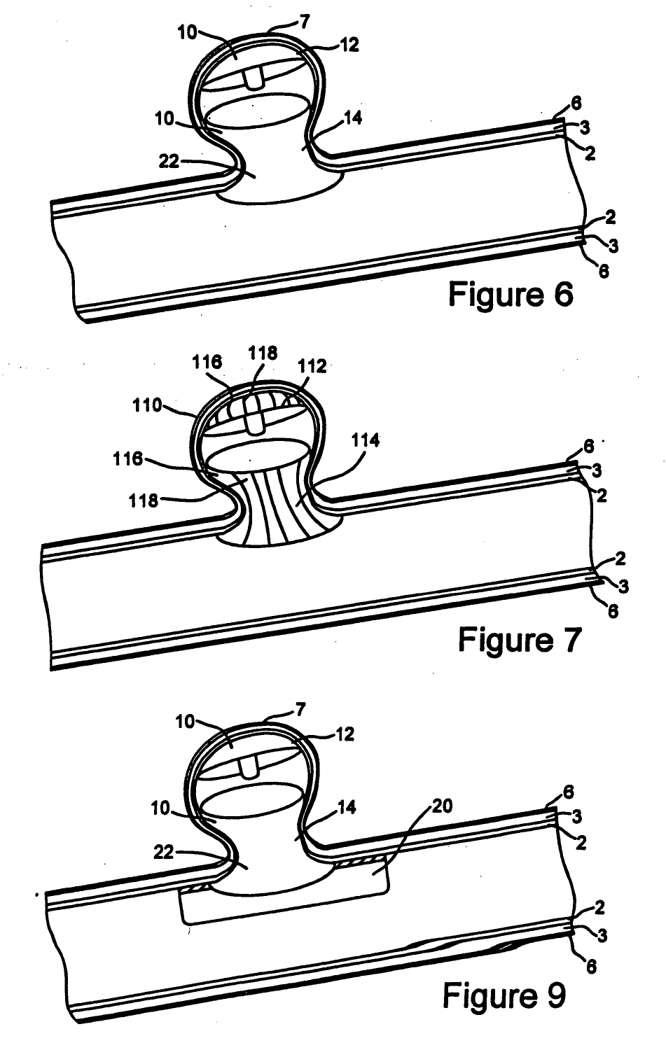

Such desirable benefits can be obtained using the preferred embodiment of an implant 10, illustrated in FIG. 6. Implant 10 can comprise a body formed of a polymeric foam or reticulated biodurable elastomeric matrix or other suitale material and can be designed to be inserted into an aneurysm through a catheter. A preferred foam is a compressible, lightweight material, chosen for ability to expand within the aneurysm to provide support to the weakened walls of the aneurysm without expanding too much and tearing the aneurysm. Additionally, in most cases for the healing process to occur, the implant 10 cannot take up the whole space of the aneurysm, as this would stop blood flow through the aneurysm which is necessary for the healing process. However, implant 10 should be sufficiently large to attenuate the pulse pressure exerted on the walls of the blood vessel to reduce the risk of further damage and leaking of the aneurysm.

More than one implant may be used for a single aneurysm. The volume of the implant, or implants, in situ, is preferably significantly less than the volume of the aneurysm, for example no more than 90 percent of the interior volume of the aneurysm, more preferably no more than 75 percent, referring to the volume of the abnormal structure outside the normal outer periphery of the host artery at the site of the aneurysm. However, the volume of an individual implant is preferably no more than about 60 percent of the aneurysm internal volume, more preferably from about 10 to about 40 percent of the aneurysm internal volume.

For the inflammatory responses to occur, there should be blood flow to the aneurysm. If the surgeon determines that the aneurysm can handle the blood flow, the surgeon will utilize the embodiments of the implant described below that allow blood flow. However, if the aneurysm is leaking, or the surgeon determines the walls of the aneurysm are too thin to handle the blood flow, the surgeon may choose an embodiment that seals off the aneurysm.

Employment of an implant that can support invasion of fibroblasts and other cells enables the implant in time to become a part of the healed aneurysm. Elastin can also be coated onto the implant providing an additional route of clot formation.

The implant can also contain a radiopaque substance for viewability by radiography or ultrasound to determine the orientation, location and other features of the implant.

Referring again to FIGS. 6 and 7 the illustrated implant 10 can be formed of a composite hydrophilically coated hydrophobic foam, as described hereinbelow or of other suitable material as is described herein, and is shaped like an inverted umbrella or a bowl with a central projection 12 upstanding in the bowl. Implant 10 has a flattened area 14 on an outer, generally convex surface 16 and has an inner generally concave surface 18. Extending upwardly from top surface 16, around the perimeter of top surface 16 are side walls 20 that curve outwardly from flattened area 14. If desired, reinforcing ribs (not shown) can be provided on inner surface 16 to increase the overall resiliency of the bowl enhancing its ability to expand to shape in situ.

In one embodiment of the present invention, the width or thickness of projection 12 is sufficient to provide structural support to the implant and enable implant 10 to be effectively manipulated by gripping the distal tip of projection 12. To this end, projection 12 may have a thickness of approximately 10 to 40 percent of the diameter defined by side walls 20. However, in application the projection may be thicker or narrower to serve desired purposes, such as support or collapsability for insertion into the catheter. In the embodiment shown, outer surface 21 of implant 10 is relatively smooth and designed to contact the majority of the inner wall of the aneurysm.

If desired, outer surfaces 16 and 21 can be coated, after fabrication of the implant. with functional agents, such as those described herein, optionally employing an adjuvant that secures the functional agents to the surfaces and to foam pores adjacent the outer surfaces, where the agents will become quickly available. Such external coating which may be distinguished from internal coatings provided within and preferably throughout the pores of a foam implant, as described herein, can comprise fibrin and/or other agents to promote fibroblast growth.

As shown in FIG. 7, implant 10 is generally circular as seen in plan. However, implant 10 may have any desired shape in plan, although symmetrical shapes such as elliptical or oval are preferred. Nevertheless, polygonal shapes such as hexagonal, octagonal or dodecagonal can be employed, if desired. Furthermore, it will be appreciated that the cross sectional shape in plan need not be geometrically regular. For example, employing a reticulated biodurable elastomeric matrix, a polymeric foam, or a comparably cleavable material, as the primary structural material of the implant, the implant can readily be trimmed to shape by the surgeon, before implantation, if desired, e.g. to fit an irregular structure within the aneurysm, possibly by making a concave, bite-shaped cutout in side walls 20.

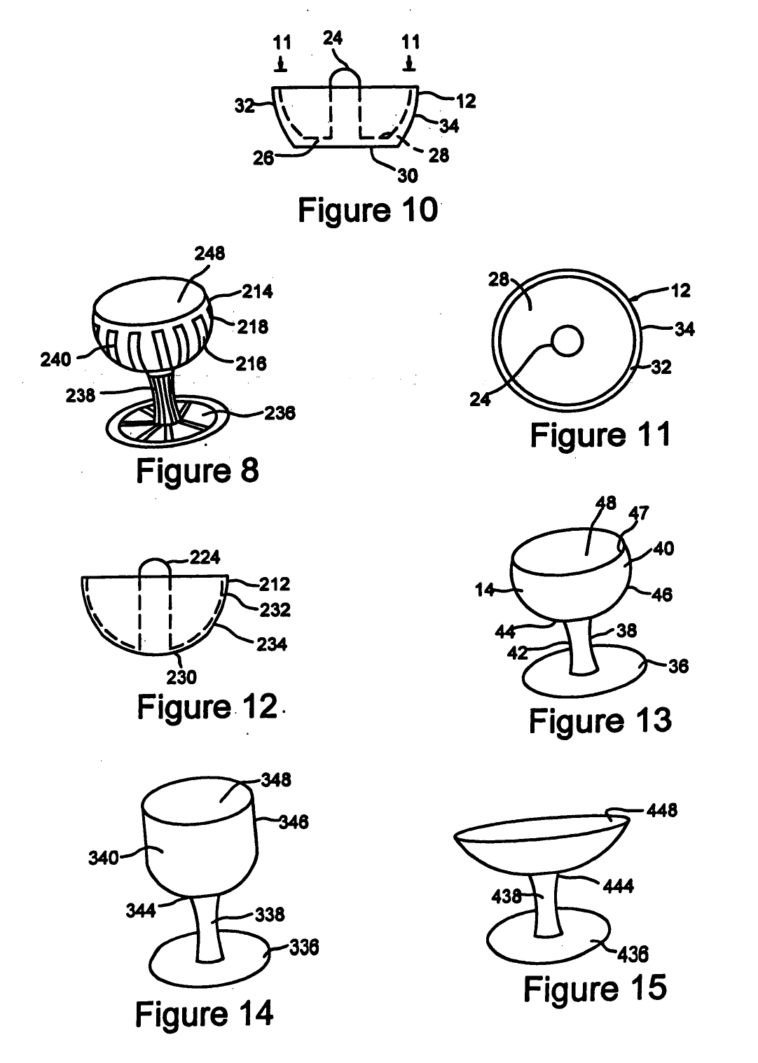

In the alternative embodiment of the invention illustrated in FIG. 8, an implant 22 is shaped much like a wine glass. More specifically, implant 14 comprises a substantially flat base 24, a column 26 and a bowl 28 Base 24 can be of any geometric shape, in the embodiment of the invention illustrated, base 24 is circular. Projecting from the center of base 24 and integral with base 24 is a column 26. The side walls 30 of column 26 can be straight, or as in the preferred embodiment, have a slight concavity. Attaching to and integral with column 26 at an end furthest from the base 24 is bowl 28. Bowl 28 has a rounded bottom 32 with sidewalls 34 extending upwardly from the rounded bottom 32 the sidewalls defining a void 36 within bowl 28. Column 26 connects to bowl 28 substantially in the center of bottom 32.

In the embodiment illustrated in FIG. 6, side walls 34 continue the curve of the rounded bottom 32, such that the side walls 34 have a convex shape. Convex walls 32 can aid in allowing blood flow within the aneurysm 7 while providing a means to accommodate pressure produced within the aneurysm. For example, instead of the pressure within the aneurysm 7 being directed toward the neck of the aneurysm, the convex shape of side walls 34 approximates the shape of the inner walls of the aneurysm in the vicinity of the neck and helps relieve pressure on those walls. Furthermore, pressure directed within bowl 28 will be diverted toward the inner surface 47 of walls 46.

Each region of implant 22 serves a particular purpose. Bowl 28 is inserted into an aneurysm and provides support to the walls of the aneurysm. Column 30 provides support to the neck of the aneurysm. Base 24 can remain outside of the aneurysm, in the lumen of the affected artery and serves to keep implant 22 in place. Further, if desired in some variants of implant 22, base 24 can be placed against the antrum of the aneurysm and the surrounding arterial wall and serve to seal off the aneurysm.

Implants 10 and 22 can be readily formed of low-cost materials and can accordingly be provided in a range or kit of different sizes and shapes from which the surgeon chooses one or more to use for a specific treatment. It is not necessary to map the aneurysm before manufacturing the implant, as is the case with the Greene et al. teaching. Such a kit of multiple sizes, e.g. from 2 to 10 different sizes and possibly also different shapes, e.g. from 2 to 6 different shapes in one or more of the particular sizes can serve a range of conditions and also is particularly valuable to have available for emergency treatments.

The implants described can be implanted by a surgeon into a particular aneurysm to be treated, singly or in combination with one or more other implants. Once an aneurysm has been identified using suitable imaging technology, such as a magnetic resonance image (MRI), computerized tomography scan (CT Scan), x-ray imaging with contrast material or ultrasound, the surgeon chooses which implant or implant or devices he feels would best suit the aneurysm, both in shape and size. The chosen implant or implants are then loaded into an intra-vascular catheter in a compressed state. The implants can be sold in a sterile package containing a pre-compressed implant that is loaded into a catheter. Alternatively, the implant can be sold in a sterile package in an expanded state, and the surgeon at the site of implantation can use a device, e.g a ring, funnel or chute that compresses the implant for loading into the catheter.

Once the implant is loaded into the catheter, the catheter is then snaked through an artery to the diseased portion of the affected artery using any of the techniques common in the art. Using the catheter the implants are then inserted and positioned within the aneurysm. Once the implant is released from its compressed state it is allowed to expand and stabilize the aneurysm.

Referring to FIG. 9, implants 10 and 22 may be seen situated in a saccular aneurysm 7. In this example, the surgeon has implanted implant 10 against the artery walls most distal from the neck 23 of the aneurysm 7, and implant 12 in the region of neck 23, and extending out of the antrum into the artery below. When properly located in situ, pursuant to the teachings of this invention, implants 10 and 12 can immediately protect the aneurysm walls from the pulsating pressure of the blood within the aneurysm which might otherwise exploit a particular weakness in the already distended aneurysm wall, resulting in catastrophic failure of the aneurysm. While the walls are so protected, the presence of implants 10 and 12, optionally including one or more pharmacologic agents borne on the or each implant, stimulates fibroblast proliferation, growth of scar tissue around the implants and eventual immobilization of the aneurysm.

Because implants are preferably each substantially smaller than the aneurysm itself, and are lightweight and can be relatively soft, having only enough resiliency to maintain their shape in situ, the risk of the implant rupturing or otherwise further aggravating the aneurysm during implantation, or subsequently, is low.

Implant 10 and implant 22 can be used in combination, wherein the projection 12 of implant 10 can fit at least partially inside void 36 of implant 22. Alternatively, as illustrated in FIG. 9, implant 10 can sit above implant 22 with little or no contact between implant 10 and implant 22.

Alternatively, as is illustrated in FIG. 10, The implants described in combination with a semicircular sectioned sheath 38, such as supplied by Boston Scientific Corporation that is applied to the wall of the artery such that the neck 23 of the aneurysm is substantially centered under the middle of the sheath 38 and blood flow to the aneurysm is cut off. Alternatively, sheath 38 can be perforated to allow blood flow into the aneurysm.

In yet another alternative embodiment of the invention illustrated in FIG. 11, implants 110 and 122 have a ribbed outer surface, the valleys between the ribs 140 providing a channel 142 for low pressure blood flow. Further, the ribbing provides reinforcement for the walls of implants 110 and 122.

Such ribbed implants could be made partially or wholly of materials other than foam. For example like an umbrella, the ribs could be formed of supportive rods radiating from and bendable toward a central strut and the area between the ribs could be a web of flexible sheeting. The ribs could be inside or outside the webs.

Referring now to FIG. 12, implant 210 is similar to implant 10 illustrated in FIG. 6 with the difference that the bottom surface 218 is rounded such that the curvature of bottom surface 218 is continuous with that of side walls 220. Bottom surface 218 and side walls 220 can form a substantially hemispheric shape.

Implants 10 and 210 are designed such that their outer surfaces 20, 220 respectively contact the inner walls of the aneurysm 1. The center projections 12, 212 can provide support and distribution of the forces exerted by the aneurysm walls. Additionally, projection 12, 212 can be used by the surgeon to further position implant 10, 210 once inserted and released from the catheter.

The inventive embodiment illustrated in FIG. 13 has a skelatal structure with open spaces between rib-like supportive members. Once inserted into the aneurysm ribs 140 can support the aneurysm walls and if desired may release one or more pharmacologic agents. Spaces such as 142 between the ribs allow for blood to flow through the aneurysm.

In an alternative embodiment illustrated in FIG. 14, side walls 346 extend straight up from rounded bottom 332 such that side walls 334 form a cylinder. In this embodiment side walls 334 can rest against the inner surface of the aneurysm.

In yet another alternative embodiment illustrated in FIG. 15, rounded bottom 432 has a less acute curve then those illustrated in FIGS. 8 and 14. In this embodiment of the invention, there are no side walls. However, it is contemplated that side walls can extend up from rounded bottom 432 if necessary to further support the walls of the aneurysm.

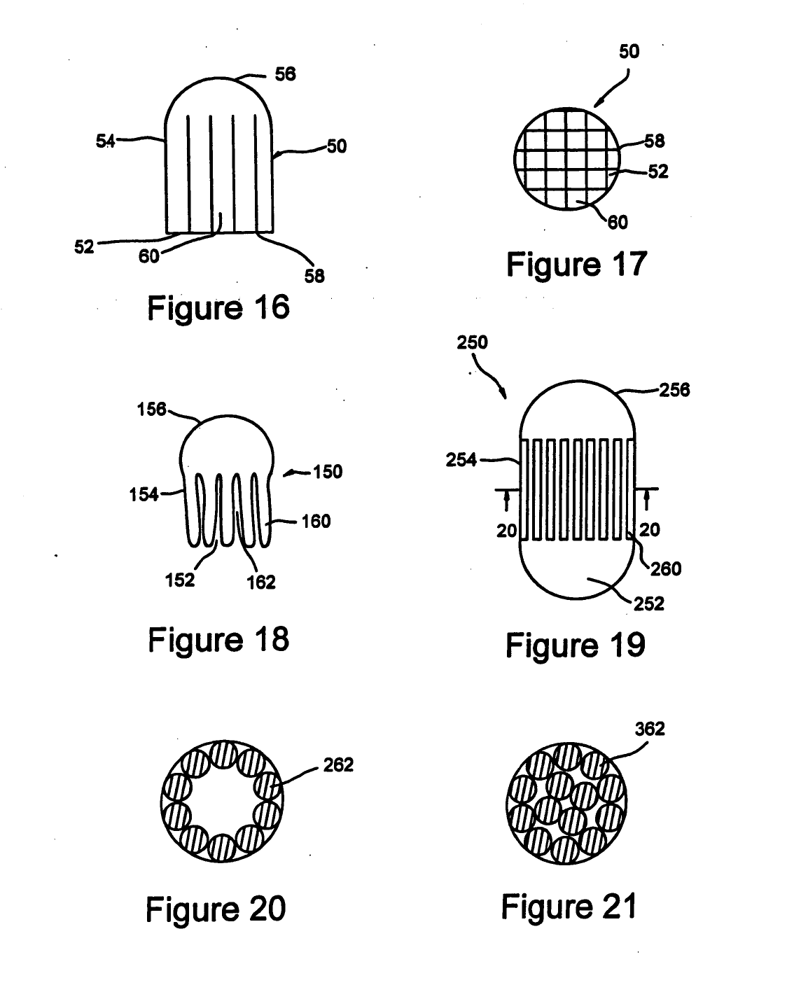

The embodiment of FIGS. 16 and 17 illustrates a bullet shaped insert 550 with a bottom 552, height 554 and top section 56 all integrally formed. The top section can be of any shape, such as pointy, flattened or as in the preferred embodiment, substantially curved. The height 554, which makes up the side walls of implant 550, is relatively straight, and bottom 552 can be of any shape, such as rounded, pointy, or as in the preferred embodiment, relatively flat. FIG. 17, a bottom view of implant 550, shows the slices 558 made in implant 550. The slices 558 create sections 60 of implant 560. These sections 560 provide increased surface area of implant 550 for more contact of the aneurysm and blood with the added chemical agents and allow implant 550 to better conform to the shape of an aneurysm as it expands.

In a similar embodiment illustrated in FIG. 18, the sections 660 of implant 650 have space 662 between them resembling the tentacles of an octopus or spaghetti.

FIG. 19 illustrates an implant 750 wherein the top 756 and bottom 752 portions are substantially solid and the side walls comprises thin strips 760. As is illustrated in FIGS. 20 and 21 which illustrates two embodiments of implant 750, the cross section of implant 750 can be hollow 762, where the side wall strips 760 are just around the perimeter of implant 750 (FIG. 20). Alternatively, as is illustrated in FIG. 21, the cross sections as viewed along lines 20-20 can be made up of strips 860 that take up substantially the entire cross section of implant 750.

FIG. 22 shows a generally tubular implant 930 formed of suitable porous elastomeric material as described elsewhere herein having an outer form 932 which is that of a right cylinder which is internally sculpted out to enhance the overall compressibility of the implant 930, with an open-ended hollow volume 934, which is also right cylindrical, or may have any other desired shape.

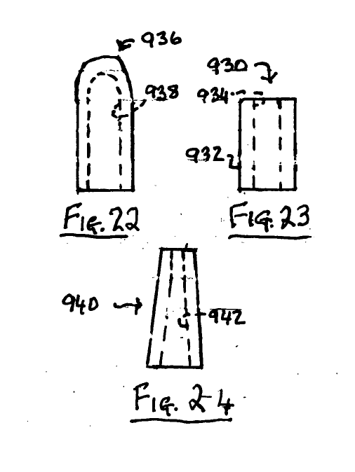

FIG. 23 illustrates a bullet-like implant 936 having a blind hollow volume 938. FIG. 24 illustrates a tapered, frusto-conical implant 940 which has an open-ended hollow volume 942. Implants 936 and 940 are generally similar to implant 930 and all three implants 930, 936 and 940 may have any desired external or internal cross-sectional shapes including circular, square, rectangular, polygonal and so on. Additional possible shapes are described hereinbelow. Alternatively, implants 930, 936 and 940 may be “solid”, with any of the described exterior shapes, being constructed throughout of porous material and lacking a hollow interior on a macroscopic scale. Desirably, any hollow interior is not closed but is macroscopically open to the ingress of fluids, i.e. fluids can directly access the macroscopic interior of the implant structure, e.g. hollows 934, 938 or 942, and can also migrate into the implant through its pore network.

While shown as largely smooth, the outer peripheries of implants 922 can have more complex shapes for desired purposes, for example, corrugated. It is contemplated that a tapered or bullet-shaped outer profile may facilitate delivery, especially of later implants arriving after a proportion of the intended group of implants has already been delivered to the target site and may offer resistance to the accommodation of newly arriving implants. For this purpose the tapered or bullet end of the implant can be oriented distally in the introducer to facilitate reception of the implant into the aneurysm volume.

The relative volumes of hollows 934, 938 and 942 are selected to enhance compressibility while still permitting implants 930, 936 and 940 to resist blood flow. Thus the hollow volumes can constitute any suitable proportion of the respective implant volume, for example in the range of from about 10 to about 90 percent with other useful volumes being in the range of about 20 to about 50 percent.

Individual ones of the shaped implants can have any one of a range of configurations, including cylindrical, conical, frustoconical, bullet-shaped, ring-shaped, C-shaped, S-shaped spiral, helical, spherical, elliptical, ellipsoidal, polygonal, star-like, compounds or combinations of two or more of the foregoing and other such configuration as may be suitable, as will be apparent to those skilled in the art, solid and hollow embodiments of the foregoing. Preferred hollow embodiments have an opening or an open face to permit direct fluid access to the interior of the bulk configuration of the implant. Other possible embodiments can be as described with reference to, or as shown in, FIG. 8, and FIGS. 10-21 of the accompanying drawings. Still further possible embodiments of shaped implant include modifying the foregoing configurations by folding, coiling, tapering, or hollowing or the like to provide a more compact configuration when compressed, in relation to the volume to be occupied by the implant in situ. Implants having solid or hollowed-out, relatively simple elongated shapes such as cylindrical, bullet-like and tapered shapes are contemplated as being particularly useful in practicing the invention.

The individual implants in an occupying body of implants employed for treating a vascular problem can be identical one with another or may have different shapes or different sizes or both. Cooperatively shaped or cooperatively sized implants may be employed to provide good packing within the target volume, if desired.

With advantage, the shaped implants can, if desired, comprise porous, elastomeric implants having a materials chemistry and microstructure as described hereinabove.

The invention also includes use of a number of implants, for example in the range of from about 2 to about 100, or in the range of from about 4 to about 30, to treat an aneurysm or other target site. Implants 930, 936 and 940, or other implants described herein may be used for this purpose.

Certain embodiments of the invention comprise reticulated biodurable elastomer products, which are also compressible and exhibit resilience in their recovery, that have a diversity of applications and can be employed, by way of example, in management of vascular malformations, such as for aneurysm control, arterio venous malfunction, arterial embolization or other vascular abnormalities, or as substrates for pharmaceutically-active agent, e.g., for drug delivery. Thus, as used herein, the term “vascular malformation” includes but is not limited to aneurysms, arterio venous malfunctions, arterial embolizations and other vascular abnormalities. Other embodiments include reticulated biodurable elastomer products for in vivo delivery via catheter, endoscope, arthroscope, laparoscope, cystoscope, syringe or other suitable delivery-device and can be satisfactorily implanted or otherwise exposed to living tissue and fluids for extended periods of time, for example, at least 29 days.

There is a need in medicine, as recognized by the present invention, for innocuous implantable devices that can be delivered to an in vivo patient site, for example a site in a human patient, that can occupy that site for extended periods of time without being harmful to the host. In one embodiment, such implantable devices can also eventually become integrated, e.g., ingrown with tissue. Various implants have long been considered potentially useful for local in situ delivery of biologically active agents and more recently have been contemplated as useful for control of endovascular conditions including potentially life-threatening conditions such as cerebral and aortic abdominal aneurysms, arterio venous malfunction, arterial embolization or other vascular abnormalities.

It would be desirable to have an implantable system which, e.g., can optionally reduce blood flow due to the pressure drop caused by additional resistance, optionally cause immediate thrombotic response leading to clot formation, and eventually lead to fibrosis, i.e., allow for and stimulate natural cellular ingrowth and proliferation into vascular malformations and the void space of implantable devices located in vascular malformations, to stabilize and possibly seal off such features in a biologically sound, effective and lasting manner.

Without being bound by any particular theory, it is thought that, in situ, hydrodynamics such as pulsatile blood pressure may, with suitably shaped reticulated elastomeric matrices, e.g., cause the elastomeric matrix to migrate to the periphery of the site, e.g., close to the wall. When the reticulated elastomeric matrix is placed in or carried to a conduit, e.g., a lumen or vessel through which body fluid passes, it will provide an immediate resistance to the flow of body fluid such as blood. This will be associated with an inflammatory response and the activation of a coagulation cascade leading to formation of a clot, owing to a thrombotic response. Thus, local turbulence and stagnation points induced by the implantable device surface may lead to platelet activation, coagulation, thrombin formation and clotting of blood.

In one embodiment, cellular entities such as fibroblasts and tissues can invade and grow into a reticulated elastomeric matrix. In due course, such ingrowth can extend into the interior pores and interstices of the inserted reticulated elastomeric matrix. Eventually, the elastomeric matrix can become substantially filled with proliferating cellular ingrowth that provides a mass that can occupy the site or the void spaces in it. The types of tissue ingrowth possible include, but are not limited to, fibrous tissues and endothelial tissues.

In another embodiment, the implantable device or device system causes cellular ingrowth and proliferation throughout the site, throughout the site boundary, or through some of the exposed surfaces, thereby sealing the site. Over time, this induced fibrovascular entity resulting from tissue ingrowth can cause the implantable device to be incorporated into the conduit. Tissue ingrowth can lead to very effective resistance to migration of the implantable device over time. It may also prevent recanalization of the aneurysm or other target site. In another embodiment, the tissue ingrowth is scar tissue which can be long-lasting, innocuous and/or mechanically stable. In another embodiment, over the course of time, for example for 2 weeks to 3 months to 1 year, implanted reticulated elastomeric matrix becomes completely filled and/or encapsulated by tissue, fibrous tissue, scar tissue or the like.

The features of the implantable device, its functionality and interaction with conduits, lumens and cavities in the body, as indicated above, can be useful in treating a number of arteriovenous malformations (“AVM”) or other vascular abnormalities. These include AVMs, anomalies of feeding and draining veins, arteriovenous fistulas, e.g., anomalies of large arteriovenous connections, abdominal aortic aneurysm endograft endoleaks (e.g., inferior mesenteric arteries and lumbar arteries associated with the development of Type II endoleaks in endograft patients).

In another embodiment, for aneurysm treatment, a reticulated elastomeric matrix is placed between a target site wall and a graft element that is inserted to treat the aneurysm. Typically, when a graft element is used alone to treat an aneurysm, it becomes partially surrounded by ingrown tissue, which may provide a site where an aneurysm can re-form or a secondary aneurysm can form. In some cases, even after the graft is implanted to treat the aneurysm, undesirable occlusions, fluid entrapments or fluid pools may occur, thereby reducing the efficacy of the implanted graft. By employing the inventive reticulated elastomeric matrix, as described herein, it is thought, without being bound by any particular theory, that such occlusions, fluid entrapments or fluid pools can be avoided and that the treated site may become completely ingrown with tissue, including fibrous tissue and/or endothelial tissues, secured against blood leakage or risk of hemorrhage, and effectively shrunk. In one embodiment, the implantable device may be immobilized by fibrous encapsulation and the site may even become sealed, more or less permanently.

In one embodiment, a patient is treated using an implantable device or a device system that does not, in and of itself, entirely fill the target cavity or other site in which the device system resides, in reference to the volume defined within the entrance to the site. In one embodiment, the implantable device or device system does not entirely fill the target cavity or other site in which the implant system resides even after the elastomeric matrix pores are occupied by biological fluids or tissue. In another embodiment, the fully expanded in situ volume of the implantable device or device system is at least 5 even 10% less than the volume of the site. In another embodiment, the fully expanded in situ volume of the implantable device or device system is at least 15% less than the volume of the site. In another embodiment, the fully expanded in situ volume of the implantable device or device system is at least 30% less than the volume of the site.

The implantable device or device system may comprise one or at least two elastomeric matrices that occupy a central location in the cavity. The implantable device or device system may comprise one or more elastomeric matrices that are located at an entrance or portal to the cavity. In another embodiment, the implantable device or device system includes one or more flexible, possibly sheet-like, elastomeric matrices. In another embodiment, such elastomeric matrices, aided by suitable hydrodynamics at the site of implantation, migrate to lie adjacent to the cavity wall.

Shaping and sizing can include custom shaping and sizing to match an implantable device to a specific treatment site in a specific patient, as determined by imaging or other techniques known to those in the art. In particular, one or at least two comprise an implantable device system for treating an undesired cavity, for example, a vascular malformation.

Some materials suitable for fabrication of the implants will now be described. Implants useful in this invention or a suitable hydrophobic scaffold comprise a porous reticulated polymeric matrix formed of a biodurable polymer that is resiliently-compressible so as to regain its shape after delivery to a biological site. The structure, morphology and properties of the elastomeric matrices of this invention can be engineered or tailored over a wide range of performance by varying the starting materials and/or the processing conditions for different functional or therapeutic uses.

The porous biodurable elastomeric matrix is considered to be reticulated because its microstructure or the interior structure comprises inter-connected open pores bounded by configuration of the struts and intersections that constitute the solid structure. The continuous interconnected void phase is the principle feature of a reticulated structure.

Preferred scaffold materials for the implants have a porous and reticulated structure with sufficient and required liquid permeability and thus selected to permit blood, or other appropriate bodily fluid, to access interior surfaces of the implants, which optionally may be drug-bearing, during the intended period of implantation. This happens due to the presence of inter-connected, reticulated open pores that form fluid passageways or fluid permeability providing fluid access all through and to the interior of the matrix for elution of pharmaceutically-active agents, e.g., a drug, or other biologically useful materials. Such materials may optionally be secured to the interior surfaces of elastomeric matrix directly or through a coating. In one embodiment of the invention the controllable characteristics of the implants are selected to promote a constant rate of drug release during the intended period of implantation. Also, the passageways may be adjusted sufficiently to permit

Any of a variety of materials meeting the foregoing requirements may be employed. A preferred foam or other porous material is a compressible, lightweight material, chosen for its structural stability in situ, its ability to support the drug to be delivered, for high liquid permeability and for an ability to substantially recover pre-compression shape and size within the bladder to provide, when loaded with appropriate substances, a reservoir of biologic agents that can be released into the blood or other fluid. Suitable materials are further described hereinbelow.

Preferred foams or hydrophobic reticulated and porous polymeric matrix materials for fabricating implants according to the invention are flexible and resilient in recovery, so that the implants are also compressible materials enabling the implants to be compressed and, once the compressive force is released, to then recover to, or toward, substantially their original size and shape. For example, an implant can be compressed from a relaxed configuration or a size and shape to a compressed size and shape under ambient conditions, e.g., at 25° C. to fit into the introducer instrument for insertion into the bladder or other suitable internal body site for in vivo delivery. Alternatively, an implant may be supplied to the medical practitioner performing the implantation operation, in a compressed configuration, for example, contained in a package, preferably a sterile package. The resiliency of the elastomeric matrix that is used to fabricate the implant causes it to recover to a working size and configuration in situ, at the implantation site, after being released from its compressed state within the introducer instrument. The working size and shape or configuration can be substantially similar to original size and shape after the in situ recovery.

Preferred scaffolds are reticulated, interconnected porous polymeric materials having sufficient structural integrity and durability to endure the intended biological environment, for the intended period of implantation. For structure and durability, at least partially hydrophobic polymeric scaffold materials are preferred although other materials may be employed if they meet the requirements described herein. Useful materials are preferably elastomeric in that they can be compressed and can resiliently recover to substantially the pre-compression state. Alternative porous polymeric materials that permit biological fluids to have ready access throughout the interior of an implant may be employed, for example, woven or nonwoven fabrics or networked composites of microstructural elements of various forms.

A partially hydrophobic scaffold is preferably constructed of a material selected to be sufficiently biodurable, for the intended period of implantation that the implant will not lose its structural integrity during the implantation time in a biological environment. The biodurable elastomeric matrices forming the scaffold do not exhibit significant symptoms of breakdown, degradation, erosion or significant deterioration of mechanical properties relevant to their use when exposed to biological environments and/or bodily stresses for periods of time commensurate with the use of the implantable device such as controlled release or elution of pharmaceutically-active agents, e.g., a drug, or other biologically useful materials over a period of time. In one embodiment, the desired period of exposure is to be understood to be at least 29 days. This measure is intended to avoid scaffold materials that may decompose or degrade into fragments for example, fragments that could have undesirable effects such as causing an unwanted tissue response.

The void phase, preferably continuous and interconnected, of the a porous reticulated polymeric matrix that is used to fabricate the implant of this invention may comprise as little as 50% by volume of the elastomeric matrix, referring to the volume provided by the interstitial spaces of elastomeric matrix before any optional interior pore surface coating or layering is applied. In one embodiment, the volume of void phase as just defined, is from about 70% to about 99% of the volume of elastomeric matrix. In another embodiment, the volume of void phase is from about 80% to about 98% of the volume of elastomeric matrix. In another embodiment, the volume of void phase is from about 90% to about 98% of the volume of elastomeric matrix.

As used herein, when a pore is spherical or substantially spherical, its largest transverse dimension is equivalent to the diameter of the pore. When a pore is non-spherical, for example, ellipsoidal or tetrahedral, its largest transverse dimension is equivalent to the greatest distance within the pore from one pore surface to another, e.g., the major axis length for an ellipsoidal pore or the length of the longest side for a tetrahedral pore. For those skilled in the art, one can routinely estimate the pore frequency from the average cell diameter in microns.

In one embodiment, the porous reticulated polymeric matrix that is used to fabricate the implant of this invention to provide adequate fluid permeability, the average diameter or other largest transverse dimension of pores is from about 50 μm to about 800 μm (i.e about 300 to 25 pores per linear inch), preferably from 100 μm to 500 μm (i.e about 150 to 35 pores per linear inch) and most preferably between 200 and 400 μm (about 80 to 40 pores per linear inch.)

In one embodiment, elastomeric matrices that are used to fabricate the scaffold part of this invention have sufficient resilience to allow substantial recovery, e.g., to at least about 50% of the size of the relaxed configuration in at least one dimension, after being compressed for implantation in the human body, for example, a low compression set, e.g., at 25° C. or 37° C., and sufficient strength and flow-through for the matrix to be used for controlled release of pharmaceutically-active agents, such as a drug, and for other medical applications. In another embodiment, elastomeric matrices of the invention have sufficient resilience to allow recovery to at least about 60% of the size of the relaxed configuration in at least one dimension after being compressed for implantation in the human body. In another embodiment, elastomeric matrices of the invention have sufficient resilience to allow recovery to at least about 90% of the size of the relaxed configuration in at least one dimension after being compressed for implantation in the human body.

In one embodiment, the porous reticulated polymeric matrix that is used to fabricate the implants of this invention has any suitable bulk density, also known as specific gravity, consistent with its other properties. For example, in one embodiment, the bulk density may be from about 0.005 to about 0.15 g/cc (from about 0.31 to about 9.4 lb/ft3), preferably from about 0.015 to about 0.115 g/cc (from about 0.93 to about 7.2 lb/ft3) and most preferably from about 0.024 to about 0.104 g/cc (from about 1.5 to about 6.5 lb/ft3).

The reticulated elastomeric matrix has sufficient tensile strength such that it can withstand normal manual or mechanical handling during its intended application and during post-processing steps that may be required or desired without tearing, breaking, crumbling, fragmenting or otherwise disintegrating, shedding pieces or particles, or otherwise losing its structural integrity. The tensile strength of the starting material(s) should not be so high as to interfere with the fabrication or other processing of elastomeric matrix. Thus, for example, in one embodiment, the porous reticulated polymeric matrix that is used to fabricate the implants of this invention may have a tensile strength of from about 700 to about 52,500 kg/m2 (from about 1 to about 75 psi). In another embodiment, elastomeric matrix may have a tensile strength of from about 700 to about 21,000 kg/m2 (from about 1 to about 30 psi). Sufficient ultimate tensile elongation is also desirable. For example, in another embodiment, reticulated elastomeric matrix has an ultimate tensile elongation of at least about 100% to at least about 500%. In one embodiment, reticulated elastomeric matrix that is used to fabricate the implants of this invention has a compressive strength of from about 700 to about 140,000 kg/m2 (from about 1 to about 200 psi) at 50% compression strain. In another embodiment, reticulated elastomeric matrix has a compressive strength of from about 7,000 to about 210,000 kg/m2 (from about 10 to about 300 psi) at 75% compression strain.

In another embodiment, reticulated elastomeric matrix that is used to fabricate the implants of this invention has a compression set, when compressed to 50% of its thickness at about 25° C., of not more than about 30%. In another embodiment, elastomeric matrix has a compression set of not more than about 20%. In another embodiment, elastomeric matrix has a compression set of not more than about 10%. In another embodiment, elastomeric matrix has a compression set of not more than about 5%.

In another embodiment, reticulated elastomeric matrix that is used to fabricate the implants of this invention has a tear strength, of from about 0.18 to about 1.78 kg/linear cm (from about 1 to about 10 lbs/linear inch).

In general, suitable porous biodurable reticulated elastomeric partially hydrophobic polymeric matrix that is used to fabricate the implant of this invention or for use as scaffold material for the implant in the practice of the present invention, in one embodiment sufficiently well characterized, comprise elastomers that have or can be formulated with the desirable mechanical properties described in the present specification and have a chemistry favorable to biodurability such that they provide a reasonable expectation of adequate biodurability.

Various reticulated hydrophobic polyurethane foams are suitable for this purpose.

In one embodiment, structural materials for the inventive porous elastomers are synthetic polymers, especially, but not exclusively, elastomeric polymers that are resistant to biological degradation, for example polycarbonate polyurethanes, polyether polyurethanes, polycarbonate polysiloxanes and the like. Such elastomers are generally hydrophobic but, pursuant to the invention, may be treated to have surfaces that are less hydrophobic or somewhat hydrophilic. In another embodiment, such elastomers may be produced with surfaces that are less hydrophobic or somewhat hydrophilic.

The invention can employ, for implanting, a porous biodurable reticulatable elastomeric partially hydrophobic polymeric scaffold material for fabricating the implant or a material. More particularly, in one embodiment, the invention provides a biodurable elastomeric polyurethane matrix which comprises a polycarbonate polyol component and an isocyanate component by polymerization, crosslinking and foaming, thereby forming pores, followed by reticulation of the foam to provide a biodurable reticulatable elastomeric product. The product is designated as a polycarbonate polyurethane, being a polymer comprising urethane groups formed from, e.g., the hydroxyl groups of the polycarbonate polyol component and the isocyanate groups of the isocyanate component. In this embodiment, the process employs controlled chemistry to provide a reticulated elastomer product with good biodurability characteristics. The foam product employing chemistry that avoids biologically undesirable or nocuous constituents therein.

In one embodiment, the starting material of the porous biodurable reticulated elastomeric partially hydrophobic polymeric matrix contains at least one polyol component. For the purposes of this application, the term “polyol component” includes molecules comprising, on the average, about 2 hydroxyl groups per molecule, i.e., a difunctional polyol or a diol, as well as those molecules comprising, on the average, greater than about 2 hydroxyl groups per molecule, i.e., a polyol or a multi-functional polyol. Exemplary polyols can comprise, on the average, from about 2 to about 5 hydroxyl groups per molecule. In one embodiment, as one starting material, the process employs a difunctional polyol component. In this embodiment, because the hydroxyl group functionality of the diol is about 2. In another embodiment, the soft segment is composed of a polyol component that is generally of a relatively low molecular weight, typically from about 1,000 to about 6,000 Daltons. Thus, these polyols are generally liquids or low-melting-point solids. This soft segment polyol is terminated with hydroxyl groups, either primary or secondary.

Examples of suitable polyol components are polyether polyol, polyester polyol, polycarbonate polyol, hydrocarbon polyol, polysiloxane polyol, poly(ether-co-ester) polyol, poly(ether-co-carbonate) polyol, poly(ether-co-hydrocarbon) polyol, poly(ether-co-siloxane) polyol, poly(ester-co-carbonate) polyol, poly(ester-co-hydrocarbon) polyol, poly(ester-co-siloxane) polyol, poly(carbonate-co-hydrocarbon) polyol, poly(carbonate-co-siloxane) polyol, poly(hydrocarbon-co-siloxane) polyol, or mixtures thereof.

Polysiloxane polyols are oligomers of, e.g., alkyl and/or aryl substituted siloxanes such as dimethyl siloxane, diphenyl siloxane or methyl phenyl siloxane, comprising hydroxyl end-groups. Polysiloxane polyols with an average number of hydroxyl groups per molecule greater than 2, e.g., a polysiloxane triol, can be made by using, for example, methyl hydroxymethyl siloxane, in the preparation of the polysiloxane polyol component.

A particular type of polyol need not, of course, be limited to those formed from a single monomeric unit. For example, a polyether-type polyol can be formed from a mixture of ethylene oxide and propylene oxide. Additionally, in another embodiment, copolymers or copolyols can be formed from any of the above polyols by methods known to those in the art. Thus, the following binary component polyol copolymers can be used: poly(ether-co-ester) polyol, poly(ether-co-carbonate) polyol, poly(ether-co-hydrocarbon) polyol, poly(ether-co-siloxane) polyol, poly(ester-co-carbonate) polyol, poly(ester-co-hydrocarbon) polyol, poly(ester-co-siloxane) polyol, poly(carbonate-co-hydrocarbon) polyol, poly(carbonate-co-siloxane) polyol and poly(hydrocarbon-co-siloxane) polyol. For example, a poly(ether-co-ester) polyol can be formed from units of polyethers formed from ethylene oxide copolymerized with units of polyester comprising ethylene glycol adipate. In another embodiment, the copolymer is a poly(ether-co-carbonate) polyol, poly(ether-co-hydrocarbon) polyol, poly(ether-co-siloxane) polyol, poly(carbonate-co-hydrocarbon) polyol, poly(carbonate-co-siloxane) polyol, poly(hydrocarbon-co-siloxane) polyol or mixtures thereof. In another embodiment, the copolymer is a poly(carbonate-co-hydrocarbon) polyol, poly(carbonate-co-siloxane) polyol, poly(hydrocarbon-co-siloxane) polyol or mixtures thereof. In another embodiment, the copolymer is a poly(carbonate-co-hydrocarbon) polyol. For example, a poly(carbonate-co-hydrocarbon) polyol can be formed by polymerizing 1,6-hexanediol, 1,4-butanediol and a hydrocarbon-type polyol with carbonate.

Furthermore, in another embodiment, mixtures, admixtures and/or blends of polyols and copolyols can be used in the elastomeric matrix of the present invention. In another embodiment, the molecular weight of the polyol is varied. In another embodiment, the functionality of the polyol is varied.

In one embodiment, the starting material of the porous biodurable reticulated elastomeric partially hydrophobic polymeric matrix contains at least one isocyanate component and, optionally, at least one chain extender component to provide the so-called “hard segment”. For the purposes of this application, the term “isocyanate component” includes molecules comprising, on the average, about 2 isocyanate groups per molecule as well as those molecules comprising, on the average, greater than about 2 isocyanate groups per molecule. The isocyanate groups of the isocyanate component are reactive with reactive hydrogen groups of the other ingredients, e.g., with hydrogen bonded to oxygen in hydroxyl groups and with hydrogen bonded to nitrogen in amine groups of the polyol component, chain extender, crosslinker and/or water.

In one embodiment, the average number of isocyanate groups per molecule in the isocyanate component is about 2. In another embodiment, the average number of isocyanate groups per molecule in the isocyanate component is greater than about 2 is greater than 2.

The isocyanate index, a quantity well known to those in the art, is the mole ratio of the number of isocyanate groups in a formulation available for reaction to the number of groups in the formulation that are able to react with those isocyanate groups, e.g., the reactive groups of diol(s), polyol component(s), chain extender(s) and water, when present. In one embodiment, the isocyanate index is from about 0.9 to about 1.1. In another embodiment, the isocyanate index is from about 0.9 to about 1.02. In another embodiment, the isocyanate index is from about 0.98 to about 1.02. In another embodiment, the isocyanate index is from about 0.9 to about 1.0. In another embodiment, the isocyanate index is from about 0.9 to about 0.98.

The elastomeric polyurethane may contain 10 to 70% by weight of hard segment, preferably 15 to 35% by weight of hard segment and may contain 30 to 85 % by weight of soft segment, preferably 50 to 80% by weight of soft segment.

Exemplary diisocyanates include aliphatic diisocyanates, isocyanates comprising aromatic groups, the so-called “aromatic diisocyanates”, and mixtures thereof. Aliphatic diisocyanates include tetramethylene diisocyanate, cyclohexane-1,2-diisocyanate, cyclohexane-1,4-diisocyanate, hexamethylene diisocyanate, isophorone diisocyanate, methylene-bis-(p-cyclohexyl isocyanate) (“H12 MDI”), and mixtures thereof. Aromatic diisocyanates include p-phenylene diisocyanate, 4,4′-diphenylmethane diisocyanate (“4,4′-MDI”), 2,4′-diphenylmethane diisocyanate (“2,4′-MDI”), 2,4-toluene diisocyanate (“2,4-TDI”), 2,6-toluene diisocyanate(“2,6-TDI”), m-tetramethylxylene diisocyanate, and mixtures thereof.

In one embodiment, the isocyanate component contains a mixture of at least about 5% to 50% by weight of 2,4′-MDI and with 50 to 95% by weight of 4,4′-MDI. Without being bound by any particular theory, it is thought that the use of higher amounts of 2,4′-MDI in a blend with 4,4′-MDI results in a softer elastomeric matrix because of the disruption of the crystallinity of the hard segment arising out of the asymmetric 2,4′-MDI structure.

In one embodiment, the starting material of the porous biodurable reticulated elastomeric partially hydrophobic polymeric matrix contains suitable chain extenders preferably for the hard segments include diols, diamines, alkanol amines and mixtures thereof. In one embodiment, the chain extender is an aliphatic diol having from 2 to 10 carbon atoms. In another embodiment, the diol chain extender is selected from ethylene glycol, 1,2-propane diol, 1,3-propane diol, 1,4-butane diol, 1,5-pentane diol, diethylene glycol, triethylene glycol and mixtures thereof. In another embodiment, the chain extender is a diamine having from 2 to 10 carbon atoms. In another embodiment, the diamine chain extender is selected from ethylene diamine, 1,3-diaminobutane, 1,4-diaminobutane, 1,5 diaminopentane, 1,6-diaminohexane, 1,7-diaminoheptane, 1,8-diaminooctane, isophorone diamine and mixtures thereof. In another embodiment, the chain extender is an alkanol amine having from 2 to 10 carbon atoms. In another embodiment, the alkanol amine chain extender is selected from diethanolamine, triethanolamine, isopropanolamine, dimethylethanolamine, methyldiethanolamine, diethylethanolamine and mixtures thereof.

In one embodiment, the starting material of the porous biodurable reticulated elastomeric partially hydrophobic polymeric matrix contains a small quantity of an optional ingredient, such as a multi-functional hydroxyl compound or other crosslinker having a functionality greater than 2, e.g., glycerol, is present to allow crosslinking. In another embodiment, the optional multi-functional crosslinker is present in an amount just sufficient to achieve a stable foam, i.e., a foam that does not collapse to become non-foamlike. Alternatively, or in addition, polyfunctional adducts of aliphatic and cycloaliphatic isocyanates can be used to impart crosslinking in combination with aromatic diisocyanates. Alternatively, or in addition, polyfunctional adducts of aliphatic and cycloaliphatic isocyanates can be used to impart crosslinking in combination with aliphatic diisocyanates.

In one embodiment, the starting material of the porous biodurable reticulated elastomeric partially hydrophobic polymeric matrix is a commercial polyurethane polymers are linear, not crosslinked, polymers, therefore, they are soluble, can be melted, readily analyzable and readily characterizable. In this embodiment, the staring polymer provides a good biodurability characteristics. The reticulated elastomeric matrix is produced by taking a solution of the commercial polymer such as polyurethane and charging it into a mold that has been fabricated with surfaces defining a microstructural configuration for the final implant or scaffold, solidifying the polymeric material and removing the sacrificial mold by melting, dissolving or subliming-away the sacrificial mold. The foam product employing a foaming process that avoids biologically undesirable or nocuous constituents therein.

Of particular interest are thermoplastic elastomers such as polyurethanes whose chemistry is associated with good biodurability properties, for example. In one embodiment, such thermoplastic polyurethane elastomers include polycarbonate polyurethanes, polyester polyurethanes, polyether polyurethanes, polysiloxane polyurethanes, polyurethanes with so-called “mixed” soft segments, and mixtures thereof. Mixed soft segment polyurethanes are known to those skilled in the art and include, e.g., polycarbonate-polyester polyurethanes, polycarbonate-polyether polyurethanes, polycarbonate-polysiloxane polyurethanes, polyester-polyether polyurethanes, polyester-polysiloxane polyurethanes and polyether-polysiloxane polyurethanes. In another embodiment, the thermoplastic polyurethane elastomer comprises at least one diisocyanate in the isocyanate component, at least one chain extender and at least one diol, and may be formed from any combination of the diisocyanates, difunctional chain extenders and diols described in detail above.

In one embodiment, the weight average molecular weight of the thermoplastic elastomer is from about 30,000 to about 500,000 Daltons. In another embodiment, the weight average molecular weight of the thermoplastic elastomer is from about 50,000 to about 250,000 Daltons.

Some suitable thermoplastic polyurethanes for practicing the invention, in one embodiment suitably characterized as described herein, include: polyurethanes with mixed soft segments comprising polysiloxane together with a polyether and/or a polycarbonate component, as disclosed by Meijs et al. in U.S. Pat. No. 6,313,254; and those polyurethanes disclosed by DiDomenico et al. in U.S. Pat. Nos. 6,149,678, 6,111,052 and 5,986,034.

Some commercially-available thermoplastic elastomers suitable for use in practicing the present invention include the line of polycarbonate polyurethanes supplied under the trademark BIONATE® by The Polymer Technology Group Inc. (Berkeley, Calif.). For example, the very well-characterized grades of polycarbonate polyurethane polymer BIONATE® 80A, 55 and 90 are soluble in THF, processable, reportedly have good mechanical properties, lack cytotoxicity, lack mutagenicity, lack carcinogenicity and are non-hemolytic. Another commercially-available elastomer suitable for use in practicing the present invention is the CHRONOFLEX® C line of biodurable medical grade polycarbonate aromatic polyurethane thermoplastic elastomers available from CardioTech International, Inc. (Woburn, Mass.). Yet another commercially-available elastomer suitable for use in practicing the present invention is the PELLETHANE® line of thermoplastic polyurethane elastomers, in particular the 2363 series products and more particularly those products designated 81A and 85A, supplied by The Dow Chemical Company (Midland, Mich.). These commercial polyurethane polymers are linear, not crosslinked, polymers, therefore, they are soluble, readily analyzable and readily characterizable.

In another embodiment of the invention the reticulated elastomeric matrix that is used to fabricate the implant can be readily permeable to liquids, permitting flow of liquids, including blood, through the composite device of the invention. The water permeability of the reticulated elastomeric matrix is from about 25 l/min./psi/cm2 to about 1000 l/min./psi/cm2, preferably from about 100 l/min./psi/cm2 to about 600 l/min./psi/cm2.

### EXAMPLE—FABRICATION OF A CROSSLINKED RETICULATED POLYURETHANE MATRIX

Aromatic isocyanates, RUBINATE 9258 (from Huntsman; comprising a mixture of 4,4′-MDI and 2,4′-MDI), are used as the isocyanate component. RUBINATE 9258 contains about 68% by weight 4,4′-MDI, about 32% by weight 2,4′-MDI and has an isocyanate functionality of about 2.33 and is a liquid at at 25° C. A polyol -1,6-hexamethylene carbonate (Desmophen LS 2391, Bayer Polymers) i.e., a diol, with a molecular weight of about 2,000 Daltons is used as the polyol component and is a solid at 25° C. Water is used as the blowing agent. The blowing catalyst is the tertiary amine 33% triethylenediamine in dipropylene glycol (DABCO 33LV supplied by Air Products). A silicone-based surfactant is used (TEGOSTAB® BF 2370, supplied by Goldschmidt). The cell-opener is ORTEGOL® 501 (supplied by Goldschmidt). A viscosity depressant (Propylene carbonate supplied by Sigma-Aldrich) is also used. The proportions of the components that are used is given in Table 1.

TABLE 1

| Ingredient | Parts by Weight |
|---|---|
| Polyol Component - Desmophen LS 2391 | 100 |
| Viscosity Depressant - Propylene carbonate | 5.76 |
| Surfactant - TEGOSTAB ® BF 2370 | 2.16 |
| Cell Opener - ORTEGOL ® 501 | 0.48 |
| Isocyanate Component RUBINATE 9258 | 53.8 |
| Isocyanate Index | 1.00 |
| Distilled Water | 2.82 |
| Blowing Catalyst | 0.44 |

The polyol Desmophen LS 2391 is liquefied at 70 oC. in an air circulation oven, and 150 gms of it is weighed into a polyethylene cup. 8.7 g of viscosity depressant (propylene carbonate) is added to the polyol and mixed with a drill mixer equipped with a mixing shaft at 3100 rpm for 15 seconds (mix-1). 3.3 g of surfactant (Tegostab BF-2370) is added to mix-1 and mixed for additional 15 seconds (mix-2). 0.75 g of cell opener (Ortogel 501) is added to mix-2 and mixed for 15 seconds (mix-3). 80.9 g of isocyanate (Rubinate 9258) is added to mix-3 and mixed for 60±10 seconds (system A).

4.2 g of distilled water is mixed with 0.66 g of blowing catalyst (Dabco 33LV) in a small plastic cup by using a tiny glass rod for 60 seconds (System B).

System B is poured into System A as quickly as possible without spilling and with vigorous mixing with a drill mixer for 10 seconds and poured into cardboard box of 9 in.×8 in.×5 in., which is covered inside with aluminum foil. The foaming profile is as follows: mixing time of 10 sec., cream time of 18 sec. and rise time of 85 sec. 2 minutes after beginning of foam mixing, the foam is place in the oven at 100-105oC. for curing for 60 minutes. The foam is taken from the oven and cooled for 15 minutes at room temperature. The skin is cut with the band saw, and the foam is pressed by hand from all sides to open the cell windows. The foam is put back in an air-circulation oven for postcuring at 100-105oC. for 5 hours.

The average pore diameter of the foam, as observed by optical microscopy, is between 150 and 350 μm.

The following foam testing is carried out in accordance with ASTM D3574. Density is measured with specimens measuring 50 mm×50 mm×25 mm. The density is calculated by dividing the weight of the sample by the volume of the specimen; a value of 2.5 lbs/ft3 is obtained.

Tensile tests are conducted on samples that are cut both parallel and perpendicular to the direction of foam rise. The dog-bone shaped tensile specimens are cut from blocks of foam each about 12.5 mm thick, about 25.4 mm wide and about 140 mm long. Tensile properties (strength and elongation at break) are measured using an INSTRON Universal Testing Instrument Model 1122 with a cross-head speed of 500 mm/min (19.6 inches/minute). The average tensile strength, measured from two orthogonal directions with respect to foam rise, is 24.64+2.35 psi. The elongation to break is approximately 215+12%.

Compressive strengths of the foam are measured with specimens measuring 50 mm×50 mm×25 mm. The tests are conducted using an INSTRON Universal Testing Instrument Model 1122 with a cross-head speed of 10 mm/min (0.4 inches/min). The compressive strength at 50% is about 12+3 psi. The compression set after subjecting the sample to 50% compression for 22 hours at 40° C. and releasing the stress is 2%.

Tear resistance strength of the foam is measured with specimens measuring approximately 152 mm×25 mm×12.7 mm. A 40 mm cut is made on one side of each specimen. The tear strength is measured using an INSTRON Universal Testing Instrument Model 1122 with a cross-head speed of 500 mm/min (19.6 inches/minute). The tear strength is determined to be about 2.9+0.1lbs/inch. In the subsequent reticulation procedure, a block of foam is placed into a pressure chamber, the doors of the chamber are closed and an airtight seal is maintained. The pressure is reduced to below 8 millitorr to remove substantially all of the air in the foam. A combustible ratio of hydrogen to oxygen gas is charged into the chamber for greater than 3 minutes. The gas in the chamber is then ignited by a spark plug. The ignition explodes the gasses within the foam cell structure. This explosion blows out many of the foam cell windows, thereby creating a reticulated elastomeric matrix structure.

Tensile tests are conducted on reticulated samples that are cut both parallel and perpendicular to the direction of foam rise. The dog-bone shaped tensile specimens are cut from blocks of foam each about 12.5 mm thick, about 25.4 mm wide and about 140 mm long. Tensile properties (strength and elongation at break) are measured using an INSTRON Universal Testing Instrument Model 1122 with a cross-head speed of 500 mm/min (19.6 inches/minute). The average tensile strength, measured from two orthogonal directions with respect to foam rise, is 23.5 psi. The elongation to break is approximately 194%.

Post reticulation compressive strengths of the foam are measured with specimens measuring 50 mm×50 mm×25 mm. The tests are conducted using an INSTRON Universal Testing Instrument Model 1122 with a cross-head speed of 10 mm/min (0.4 inches/min). The compressive strength at 50% is about 6.5 psi.

One possible material for use in the present invention comprises a resiliently compressible composite polyurethane foam comprising a hydrophilic foam coated on and throughout the pore surfaces of a hydrophobic foam scaffold. One suitable such material is the composite foam disclosed and claimed in Thomson United States patent application publication number 20020018884 assigned to Hydrophilix, LLC., U.S. Pat. No. 6,617,014 and in international patent publication number WO 01/74582 (Applicant: Hydrophilix, LLC, published Oct. 11, 2001), the entire disclosures of each of which patent applications are hereby incorporated herein by reference thereto. The hydrophobic foam provides support and resilient compressibility enabling the desired collapsing of the implant for delivery and reconstitution in situ.

The hydrophilic foam can be used to carry a variety of therapeutically useful agents, for example, agents that can aid in the healing of the aneurysm, such as elastin, collagen or other growth factors that will foster fibroblast proliferation and ingrowth into the aneurysm, agents to render the foam implant non-thrombogenic, or inflammatory chemicals to foster scarring of the aneurysm. Furthermore the hydrophilic foam, or other agent immobilizing means, can be used to carry genetic therapies, e.g. for replacement of missing enzymes, to treat atherosclerotic plaques at a local level, and to release agents such as antioxidants to help combat known risk factors of aneurysm.

Pursuant to the present invention it is contemplated that the pore surfaces may employ other means besides a hydrophilic foam to secure desired treatment agents to the hydrophobic foam scaffold.

The agents contained within the implant can provide an inflammatory response within the aneurysm, causing the walls of the aneurysm to scar and thicken. This can be accomplished using any suitable inflammation inducing chemicals, such as sclerosants like sodium tetradecyl sulphate (STS), polyiodinated iodine, hypertonic saline or other hypertonic salt solution. Additionally, the implant can contain factors that will induce fibroblast proliferation, such as growth factors, tumor necrosis factor and cytokines.

An alternative embodiment is also contemplated by the inventor wherein the target aneurysm is identified and imaged, one or more customized implants can be provided which is a close fit to the aneurysm. Such customized implants can be made, for example, by the methods described by Greene, Jr. et al., the entire disclosure of which is hereby incorporated herein by this reference thereto. However, in contrast to the teaching of Greene, Jr. et al., such customized implant, which may be a composite of two or three or more separately delivered implants, also includes a pharmacologic agent to promote fibroblast invasion, and sealing of the aneurysm with scar tissue, as described herein, and is preferably also formed sufficiently smaller than the aneurysm to permit limited blood flow around the aneurysm.

It is further contemplated that medical facilities performing aneurysm treatments can employ computer controlled systems on site to make suitable implants. Thus, an aneurysm can be imaged and the image loaded into the computer. The computer will make a virtual image of the aneurysm. The surgeon can then choose the type of implant he desires, load a universal form into the machine and the system will size and shape that form according to the image of the aneurysm and the surgeons entered specifications.

In another aspect, the invention provides a method for the treatment or prevention of endoleaks from an implanted endovascular graft into a target vascular site, for example an aneurysm, or an abdominal aortic aneurysm. the method comprising delivering a number of porous elastomeric implants in a compressed state, into the target site. The number of implants can be in the range of from about 2 to about 100, for example from about 4 to about 30, or any other suitable number.

Usefully, the implants can occlude feeder vessels that open into the aneurysm site, to control what are known as Type II endoleaks which may be caused by retrograde flow from collateral arteries. To this end, the perigraft space between the endograft and the aneurysm can be filled or substantially filled with a number of implants that are relatively small compared with the target site. In one embodiment, the invention provides for at least some of the delivered implants to be partially, but not fully, expanded in situ, retaining some of their resilient compression as residual compression.

Such an endoleak treatment method may be performed post-operatively, at an appropriate period, perhaps days, weeks or months after implantation of an endograft. Alternatively, if suitable criteria are met, endoleak treatment may be effected prophylactically at the time of endograft implantation.

The invention also provides apparatus for performing the method, the apparatus comprising an introducer for delivering implants and a suitable number of implants for delivery to the target site.

Although the invention has been described in terms of its applicability to aneurysms, it will be understood that the devices and methods of the invention may be useful for other purposes including the treatment of tumors and the treatment of lesions such as arteriovenous malformations (AVM), arteriovenous fistula (AVF), uncontrolled bleeding and the like

The entire disclosures of each of the United States patents or patent applications, foreign or international patent publications, or other publications, or unpublished patent applications that are referenced in this specification, or elsewhere in this patent application, are hereby incorporated herein by each respective specific reference made thereto.

In one embodiment the reticulated biodurable elastomeric matrix can have a larger dimension of from about 1 to about 100 mm optionally from about 3 to 50 mm, when a plurality of relatively small implants is employed.

While illustrative embodiments of the invention has been described, it is, of course, understood that various modifications of the invention will be obvious to those of ordinary skill in the art. Such modifications are within the spirit and scope of the invention which is limited and defined only by the appended claims.

We claim:

1. An aneurysm treatment device for in situ treatment of aneurysms in mammals, optionally humans, the treatment device comprising at least one resiliently collapsible implant collapsible from a first, expanded configuration wherein the implant can support the wall of an aneurysm to a second collapsed configuration wherein the collapsible implant is deliverable into the aneurysm, and wherein the implant does not completely fill the aneurysm.

2. An aneurysm treatment device according to claim 1 wherein the implant has sufficient resilience, or swellability, to return to an expanded configuration within the lumen of the aneurysm.

3. An aneurysm treatment device according to claim 1 wherein the implant is configured so that hydraulic forces within the aneurysm tend to urge the implant against the aneurysm wall.

4. An aneurysm treatment device according to claim 1 wherein the collapsible implant comprises a spreadable portion and a projecting portion, the spreadable portion capable of resting against and providing support to an inner wall of the aneurysm, the projecting portion being integral with the spreadable portion and being capable of being gripped for insertion and positioning of the implant.

5. An aneurysm treatment device according to claim 1 wherein the implant comprises a resiliently compressible polymeric foam.

6. An aneurysm treatment device according to claim 5 wherein the foam member comprises a hydrophobic foam scaffold member coated on the pore surfaces of the foam, within the foam body, to be hydrophilic, optionally with a coating of hydrophilic foam material.

7. An aneurysm treatment device according to claim 6 wherein the foam member comprises a hydrophobic foam scaffold member coated on the pore surfaces of the foam and throughout the pores of the foam with a hydrophilic foam, and wherein the hydrophilic foam carries a pharmacologic agent, optionally fibrin or a fibroblast growth factor, or both.

8. An aneurysm treatment device according to claim 1 comprises a pair of implants cooperable to stabilize the aneurysm.

9. An aneurysm treatment device according to claim 8 wherein one implant, optionally a generally wine glass-shaped implant, can be seated in the neck of the aneurysm and has a spreading portion spreading into the aneurysm to support the aneurysm wall adjacent the antrum and the other implant, optionally a generally mushroom-shaped implant, can ride in the aneurysm and has a spreading portion to support the aneurysm wall opposite the neck of the aneurysm.

10. An aneurysm treatment device according to claim 1 wherein the implant further comprises one or more bioactive materials selected from the group consisting of elastin, growth factors capable of fostering fibroblast proliferation, pharmacologic agents, sclerotic agents, inflammatory substances, genetically acting therapeutics and genetically engineered therapeutics.

11. An aneurysm treatment device according to claim 1 comprising a set of multiple ones of the implant, the set comprising a range of different sizes of the implant, optionally from 2 to about 10 different sizes, and a range of different shapes of the implant, optionally from 2 to about 6 different shapes in one or more of the sizes.

12. An aneurysm treatment device according to claim 1 wherein the spreading portion of the implant comprises a convex outer surface to contact the aneurysm wall and a concave inner surface.

13. An aneurysm treatment device according to claim 1 wherein implant comprises a foam member having an inner surface and an outer surface, the outer surface having areas of elevations and depression capable of allowing blood flow between the inner wall of the aneurysm and the outer surface of the foam member.

14. An aneurysm treatment device according to claim 1 wherein the implant is porous and permits blood flow into the interior of the implant.

15. An aneurysm treatment device according to claim 1 wherein the implant comprises a reticulated biodurable elastomeric matrix.

16. An aneurysm treatment device according to claim 1 wherein the implant comprises a reticulated biodurable elastomeric matrix and the implant exhibits resilient recovery from compression.

17. An aneurysm treatment device according to claim 1 comprising multiple implants wherein each implant has the shape of a cylinder, a right cylinder, is bullet-shaped, is bullet-shaped with a blind hollow volume, has a tapered, frusto-conical shape optionally with an open-ended hollow volume with a circular, square, rectangular, polygonal cross-section.

18. A method of treating an aneurysm comprising the steps of:
a) imaging an aneurysm to be treated to determine its size and topography;
b) selecting an aneurysm treatment device according to claim 1 for use in treating the aneurysm; and
c) implanting the aneurysm treatment device into the aneurysm.

19. A method according to claim 18 further comprising:
d) loading the aneurysm treatment device into a catheter;
e) threading the catheter through an artery to the aneurysm; and
f) positioning and releasing the aneurysm treatment device in the aneurysm.

20. A method of treating an aneurysm comprising the steps of:
a) imaging an aneurysm to be treated to determine its size and topography;
b) constructing an aneurysm treatment device to be shaped to fill the aneurysm in situ and to be deliverable via a catheter, the aneurysm treatment device optionally being resiliently collapsible or swellable to expand to shape in situ and including in the aneurysm treatment device a pharmacologic agent for delivery within the aneurysm;
c) implanting the aneurysm treatment device into the aneurysm.

21. A method according to claim 18 wherein the aneurysm treatment device is configured to permit limited blood access between the implant and the aneurysm wall, optionally without significantly pulsing the aneurysm wall.

22. A method for the treatment or prevention of endoleaks from an implanted endovascular graft into a target vascular site, optionally an aneurysm, the method comprising delivering a number of porous and/or reticulated elastomeric implants in a compressed state, into the target site.

23. A method according to claim 22 wherein the number of implants is in the range of from about 2 to about 100.

24. A method according to claim 23 wherein the implants comprise reticulated biodurable elastomeric matrices.
